(19) United States
(12) Patent Application Publication
(10) Pub. No.: US 2010/0249538 A1
(43) Pub. Date: Sep. 30, 2010

(54) PRESENTATION MEASURE USING NEUROGRAPHICS

(75) Inventors: Anantha Pradeep, Berkeley, CA (US); Robert T. Knight, Berkeley, CA (US); Ramachandran Gurumoorthy, Berkeley, CA (US)

Correspondence Address: Weaver Austin Villeneuve & Sampson LLP, P.O. BOX 70250, OAKLAND, CA, 94612-0250, US

(73) Assignee: NeuroFocus, Inc., Berkeley, CA (US)

(21) Appl. No.: 12/410,372

(22) Filed: Mar. 24, 2009

(51) Int. Cl. A61B 5/00

(52) U.S. Cl. 600/301

(57) ABSTRACT

Presentation of materials such as advertising and marketing materials is evaluated and/or dynamically modified using neurographical data. User images, video, and audio, etc. are analyzed when a user is presented with stimulus materials. User data such as a user image is matched with a neurographical aggregate to identify user information and emotional state. The neurographical aggregate identifies actions and/or additional stimulus material for presentation to the user. User information and emotional state also allow evaluation of the presented materials.

### TECHNICAL FIELD

The present disclosure relates to neurographics. More particularly, the present disclosure relates to determining presentation measures using neurographics.

### DESCRIPTION OF RELATED ART

A variety of conventional systems are available for evaluating the presentation of an advertisement. User may complete survey forms or participate in focus groups after viewing an advertisement, reading text messages, seeing a billboard, etc. Users may themselves be surveyed to determine what they watched and for how long. In some examples, cameras and video recorders are placed near advertisements including billboards to monitor viewer activity and attention span. However, conventional mechanisms have a variety of limitations in evaluating the presentation of stimulus material.

Consequently, it is desirable to provide improved mechanisms for evaluating the presentation of stimulus materials such as advertising, entertainment, etc.

### BRIEF DESCRIPTION OF THE DRAWINGS

The disclosure may best be understood by reference to the following description taken in conjunction with the accompanying drawings, which illustrate particular example embodiments.

FIG. 1 illustrates one example of a system for evaluating the presentation of stimulus material using neurographics.

FIG. 2 illustrates example neurographical aggregates for various profiles.

FIG. 3 illustrates one example of generating neurographical aggregates.

FIG. 4 illustrates one example of a neurographical aggregate database.

FIG. 5 illustrates one example of a system for evaluating neurological aggregates.

FIG. 6 illustrates one example of a technique for performing market matching and stimulus presentation using neurographics.

FIG. 7 provides one example of a system that can be used to implement one or more mechanisms.

### DESCRIPTION OF PARTICULAR EMBODIMENTS

Reference will now be made in detail to some specific examples of the present invention including the best modes contemplated by the inventors for carrying out the invention. Examples of these specific embodiments are illustrated in the accompanying drawings. While the invention is described in conjunction with these specific embodiments, it will be understood that it is not intended to limit the invention to the described embodiments. On the contrary, it is intended to cover alternatives, modifications, and equivalents as may be included within the spirit and scope of the invention as defined by the appended claims.

For example, the techniques and mechanisms of the present invention will be described in the context of particular types of media. However, it should be noted that the techniques and mechanisms of the present invention apply to a variety of different types of media. In the following description, numerous specific details are set forth in order to provide a thorough understanding of the present invention. Particular example embodiments of the present invention may be implemented without some or all of these specific details. In other instances, well known process operations have not been described in detail in order not to unnecessarily obscure the present invention.

Various techniques and mechanisms of the present invention will sometimes be described in singular form for clarity. However, it should be noted that some embodiments include multiple iterations of a technique or multiple instantiations of a mechanism unless noted otherwise. For example, a system uses a processor in a variety of contexts. However, it will be appreciated that a system can use multiple processors while remaining within the scope of the present invention unless otherwise noted. Furthermore, the techniques and mechanisms of the present invention will sometimes describe a connection between two entities. It should be noted that a connection between two entities does not necessarily mean a direct, unimpeded connection, as a variety of other entities may reside between the two entities. For example, a processor may be connected to memory, but it will be appreciated that a variety of bridges and controllers may reside between the processor and memory. Consequently, a connection does not necessarily mean a direct, unimpeded connection unless otherwise noted.

Overview

Presentation of materials such as advertising and marketing materials is evaluated and/or dynamically modified using neurographical data. User images, video, and audio, etc. are analyzed when a user is presented with stimulus materials. User data such as a user image is matched with a neurographical aggregate to identify user information and emotional state. The neurographical aggregate identifies actions and/or additional stimulus material for presentation to the user. User information and emotional state also allow evaluation of the presented materials.

### Example Embodiments

Conventional mechanisms for evaluating user responses to stimulus material and modifying stimulus materials for presentation to a user typically rely on user input such as surveys and focus groups. Users convey thoughts and feelings about an advertisement, a program, a banner, a billboard, a shelf display, interior decorations, etc., by responding to questionnaires. However, these responses provide limited information on actual user thoughts and feelings. A variety of semantic, syntactic, metaphorical, cultural, social and interpretive biases and errors prevent accurate and repeatable evaluation.

Online advertisers have additional information about how many individuals click on an advertisement, when the individuals view it, and may even have demographic information about the users who select the advertisement. However, user response information is again limited. Some billboards are equipped with cameras that allow monitoring of viewers passing by to determine how many look at the billboard and for how long. The cameras also may gather detail about viewers including gender and approximate age. The cameras use software to determine that a person is standing in front of a billboard and analyze factors such as cheekbone height and the distance between the nose and the chin to guess a person's gender and age.

These billboards may be inside malls, elevators, casinos, restaurants or may be located outside. However, insights on user responses to advertisements and other stimulus material are again limited.

Consequently, the techniques of the present invention provide improved evaluation of media and dynamic adjustment of stimulus presentation based on neurographics. The system may be implemented on a billboard with a camera, a computer with a webcam, or a business place with a microphone. According to particular embodiments, neurographical aggregates are created for different categories of people for a variety of emotional states. According to particular embodiments, neurographical aggregates are created across eye colors, skin tones, genders, and age groups for a range of emotions such as happiness, surprise, fear, anger, sadness, disgust, awe, and disinterest (or neutral emotions).

In particular embodiments, 40 teenage boys having a light skin tone and brown eyes are recorded expressing various emotions. Facial expressions as well as body gestures and posture may be recorded. The recordings of the 40 teenage boys are combined into a neurographical aggregate. In a particular example, one neurographical aggregate is a combination of the images of 40 teenage boys having light skin tone and brown eyes expressing awe. In some examples, stimulus material and actions particularly effective for the 40 teenage boys having light skin tone and brown eyes in various emotional states are associated with each of the aggregates. Hairstyles and clothing styles may also be used as characteristics in developing aggregates. In some examples, a more somber advertisement may be presented to an individual expressing sadness. A more dynamic advertisement may be presented to younger individuals expressing neutral emotions.

According to particular embodiments, neuro-response data is collected and analyzed when generating aggregates to determine actual emotional responses to stimulus material. Stimulus material and marketing categories deemed particularly effective for particular aggregates are identified and associated with the aggregates. For example, action movie advertisements may be associated with teenage boys who express happiness, surprise, or awe. Reactions to action movie advertisements may again be measured to determine effectiveness of the action movie advertisements. If the reaction is an expression of happiness or awe, additional action movie advertisements may be presented.

According to particular embodiments, different material is presented based on characteristics of a neurographic profile. In particular embodiments, the system may recognize that the user is current with the latest fashion trends and may present different advertisements using that information along with facial emotional expressions. In other embodiments, the system may recognize that a viewer has a child in a stroller and has an expression matching a particular aggregate. The system may present child-care or babysitting offers appropriate for parents with young children.

According to various embodiments, it is recognized that some individuals, particularly individuals over 40 years of age, can simultaneously process only three visual elements while others can process five visual elements. In particular embodiments, the three visual elements may be three portions of a video advertisement on a digital billboard. The number of visual elements simultaneously processed can decrease with age. According to various embodiments, the number of stimultaneous visual elements can be automatically selected based on neurographical information.

In still other examples, the amount of detail a user is drawn to is determined using neurographical aggregates. According to various embodiments, it is recognized that particular populations, groups, and subgroups of people are drawn to detail while others may ignore detail. The amount of detail includes intricacies in the design of a product. According to various embodiments, marketing categories presented to a user can be automatically selected based on neurographical information.

Although facial emotion expression is described, it should be noted that a wide variety of characteristics may be evaluated using neurographical aggregates. According to various embodiments, the amount of dynamism needed in media to elicit a response may be determined using neurographical aggregates. In particular embodiments, younger individuals with more rapidly changing expressions may require more dynamism and changing visual elements in order to hold attention. The number of distractors, number of repetitions, or the length of a message may also be varied based on neurographical aggregates.

The targeted stimulus material may be presented in a variety of locations using a number of different media. Material may be presented on digital billboards, checkout displays, waiting room and subway station screens, etc. Material may be dynamically modified and adjusted based on updated neurographical information obtained while the user is presented with stimulus material.

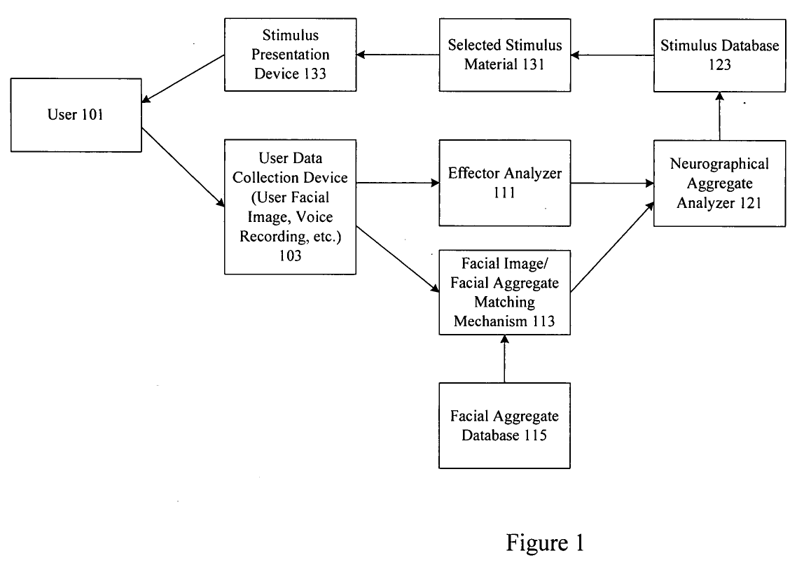

FIG. 1 illustrates one example of a system for using neurographical aggregates. According to various embodiments, a user 101 is presented with stimulus from a stimulus presentation device 133. In particular embodiments, the stimulus presentation device 133 is a billboard, display, monitor, screen, speaker, etc., that provides stimulus material to a user. Continuous and discrete modes are supported. According to various embodiments, the stimulus presentation device 133 also has protocol generation capability to allow intelligent modification of stimuli provided to the user based on neurographical feedback. User data is obtained using a user data collection device 103. The user data collection device 103 may be a video camera, audio recorder, digital camera, etc. According to particular embodiments, the user data collection device 103 monitors user expressions, posture, gestures, and other characteristics associated with the user. For example, the user data collection device 103 may monitor user hairstyles, clothing, eyebrow shape, accessories, jewelry, as well as whether the user is alone or in a group.

According to particular embodiments, an effector analyzer 111 and facial image/facial aggregate matching mechanism 113 analyze user data. In particular embodiments, the effector analyzer 111 and the facial image/facial aggregate matching mechanisms 113 may be integrated or separate. Effector analyzer 111 may track eye movements and reaction time to various stimulus materials. In some embodiments, autonomic nervous system measures such as pupillary dilation may also be tracked using a camera. According to particular embodiments, facial image/facial aggregate matching mechanism analyzes characteristics and expressions of a user and matches one or more images with aggregates in a facial aggregate database 115.

In particular embodiments, the facial aggregate database 115 includes aggregates of faces, gestures, postures, etc. The facial aggregate database 115 may include aggregated images of 100 Latino middle aged women with a wide range of emotional expressions, 100 white teenaged girls with a range of emotional expressions, 100 wealthy businessmen with a wide range of emotional expressions, etc. According to particular embodiments, a neurographical aggregate analyzer 121 determines market categories and/or stimulus materials from stimulus database 123 for presentation to a particular user 101 having a particular neurographical profile at a given time. Selected stimulus materials 131 are provided to the stimulus presentation device 133 to provide initial or dynamically updated target materials to the user 101. In some examples, if a neurographical aggregate analyzer 121 determines that a viewer is exhibiting a neutral response even during the middle of a video advertisement, a new advertisement may be presented.

According to particular embodiments, neurographical aggregates are generated across different age groups, skin tones, eye colors, hairstyles, genders, etc. It is recognized that different groups respond differently to the same stimulus. For example, one group may view an advertisement for 20 seconds while another group may only view the advertisement for 5 seconds, but the different groups may actually both as likely to purchase a product because of the advertisement. One group may process a banner more quickly before showing a positive neurographical response while another group may examine the details of the banner for a longer length of time before showing any neurographical response.

It is further recognized that many individuals may not fit an aggregate into which they are categorized. For example, a retired woman may be just as interested in action video games as a teenage boy. According to particular embodiments, a user may voluntarily offer to provide neurographical information and personalized preferences. When a particular user is detected, personalized advertising may be provided based on particular emotions exhibited by the user. Expressions may be used to select or reject additional stimulus material.

However, in many instances, personalized neurographical information is not available. Consequently, neurographical aggregates are analyzed to determine stimulus material that would be of interest to a user. According to particular embodiments, the neurographical aggregate analyzer also recognizes when a neurographical aggregate matched with a user is not the best match. For example, the user may exhibit a neutral or negative emotional response to a series of offers presented. In particular embodiments, more generic stimulus material would then provided to the user. In other examples, another match with a neurographical aggregate may be attempted with a focus on different characteristics such as clothing and hairstyle instead of eye mouth distance or skin tone.

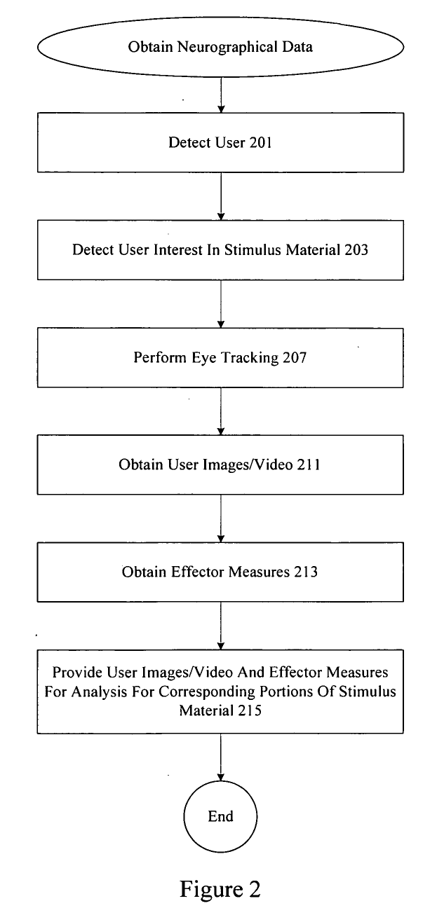

FIG. 2 illustrates one example of obtaining a neurographical data. At 201, a user is detected. According to various embodiments, a user may be detected based on noise levels, heat, eye contact, etc. At 203, user interest in stimulus material is detected. User interest may be determined by measuring sustained eye contact with a billboard. According to particular embodiments, a message may be presented indicating to the user that targeted material is available if the user is interested. In particular embodiments, a message may be presented allowing a user to opt out of having personalized information presented using neurographics. If the user shows interest, at 207, eye tracking may be performed if possible. At 211, user media is recorded. Effector measures 213 may be obtained as part of the recording of media. At 215, media such as images, video, and audio are provided for analysis. The user media may be mapped with particular portions of stimulus material viewed by a user. For example, eye tracking data may indicate that the user is paying attention to the left half of a billboard. Neurographical data is provided for analysis and modification of the left half of the billboard.

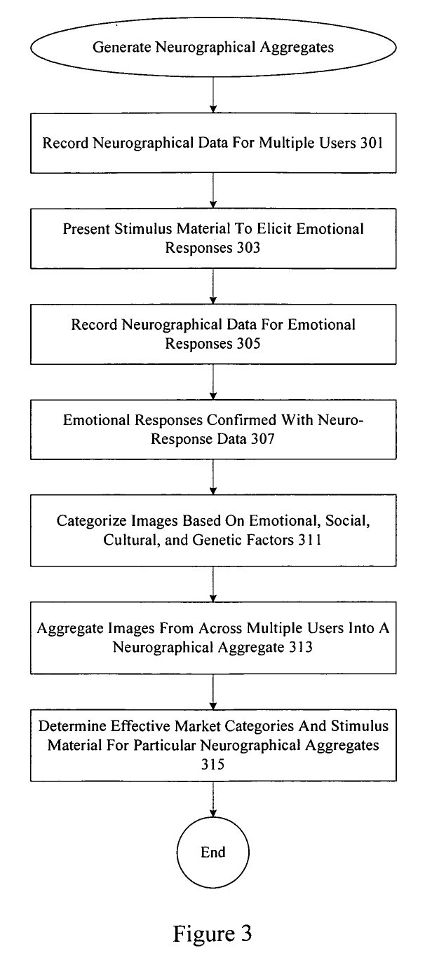

FIG. 3 illustrates one example of generating neurographical aggregates. At 301, neurographical data is recorded for multiple users across a variety of characteristics. For example, neurographical data for numerous users may be recorded using cameras. Neurographical data may be obtained for thousands of subjects and categorized based on eye color, skin tone, face shape, age, gender, etc. At 303, stimulus material is presented to elicit emotional responses. According to particular embodiments, stimulus material may be video, audio, text, conversations, products, offers, events, etc. At 305, neurographical data is recorded for each of the emotional responses elicited. In particular embodiments, emotional responses include happiness, surprise, fear, anger, sadness, disgust, awe, and disinterest (or neutral emotions).

According to particular embodiments, emotional responses are confirmed using neuro-response data collected using mechanisms such as EEG and fMRI at 307. For example, a neutral response may be confirmed by lack of any change in neuro-response activity. Happiness may be confirmed by evidence of the occurrence or non-occurrence of specific time domain difference event-related potential components (like the DERP) in specific brain regions. In particular embodiments, a large number of items eliciting known responses are presented to subjects to obtain neurographical data for various emotions.

According to various embodiments, images are categories based on emotional, social, cultural, and genetic factors at 311. Although images are described, it should be noted that a range of different media may be used and categorized. In some examples, numerous images of teenage boys having brown eyes and beige skin tones with happy emotional expressions are combined to form an aggregated image. According to particular embodiments, neurographical aggregates are created for a large number of categories. In some examples, mothers with a child having long hair, green eyes, and darker skin tones and emotional expressions of awe are combined to form an aggregated image for the noted characteristics at 313.

At 315, market categories and stimulus material effective for particular neurographical aggregates are determined. For example, a man wearing a baseball cap and having a neutral expression may find ticket offers for sporting events particularly interesting. An older women having an expression of fear may be directed to offers for secure savings accounts. According to particular embodiments, the neurographical aggregates can be used to provide stimulus material to a viewer and to evaluate the effectiveness of stimulus material being presented to the user.

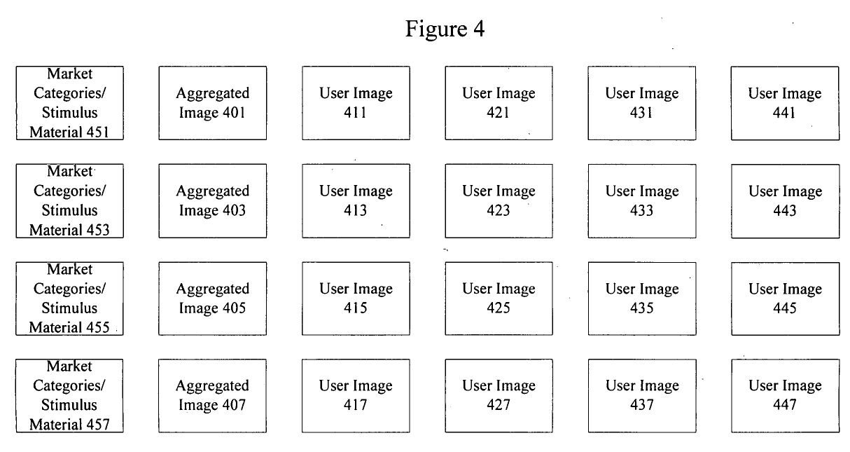

FIG. 4 illustrates one example of neurographical aggregates. According to particular embodiments, aggregated image 401 corresponds to a set of characteristics such as brown eyes, lighter skin tone, female, middle-aged, slim, long hair, etc. User images 411, 421, 431, and 441 may be combined to form the aggregated image 401. According to particular embodiments, hundreds of images may be combined to form an aggregate image 401. Similarly, user images 413, 423, 433, and 443 may be combined to form the aggregated image 403. User images 415, 425, 435, and 445 may be combined to form the aggregated image 405. User images 417, 427, 437, and 447 may be combined to form the aggregated image 407.

Aggregates may be formed by identifying reference points and vectors user images. Reference points and vectors may correspond to particular facial features, edges, high contrast areas, etc. For example, multiple reference points and vectors may be used to identify the contours of a brow. Various landmark based and image based approaches can be used. Landmark based approaches use corresponding pairs of points and line segments in source and target images. Image based approaches identify features based on pixel intensities and variations. Eye, nose, and mouth detection algorithms can be applied to identify corresponding features. In one example, bilinear transformation maps quadrangles created by reference points in user images to quadrangles created by corresponding reference points in other images. Coordinate transformation may also be applied to warp the user images into an average of the user images to generate an aggregated image 401.

According to particular embodiments, a neurographical aggregate database also includes information identifying market categories and stimulus material appropriate for particular aggregated images and corresponding sets of characteristics. For example, market categories and stimulus material 451 correspond to aggregated image 401, market categories and stimulus material 453 correspond to aggregated image 403, market categories and stimulus material 455 correspond to aggregated image 405, and market categories and stimulus material 457 correspond to aggregated image 407.

The market categories and stimulus material may be generated when aggregate images and user images are obtained. The market categories and stimulus material may also be generated and/or updated dynamically as users or viewers respond to presented stimulus in a positive or negative fashion.

Although images are depicted, neurographical aggregates may include aggregated voice recordings, aggregated videos, aggregated smells, etc.

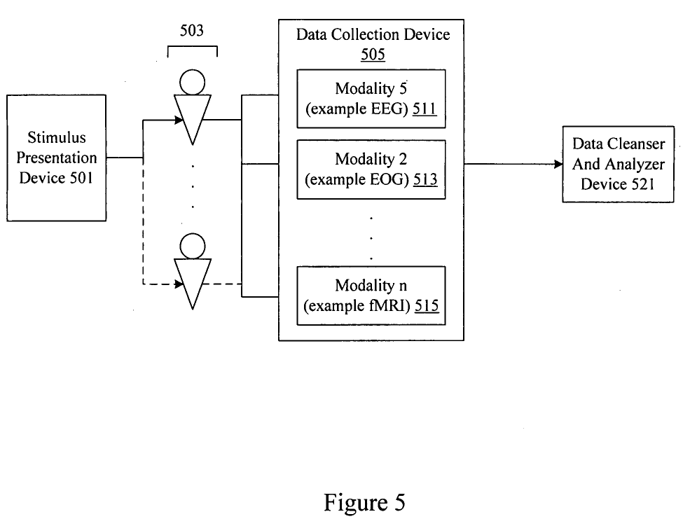

FIG. 5 illustrates one example of a system for determining market categories and stimulus material for neurographical aggregates. Neuro-response data can be collected and analyzed to determine appropriate market categories and stimulus materials for particular neurographical aggregates.

According to various embodiments, a system for evaluating neurological profiles includes a stimulus presentation device 501. In particular embodiments, the stimulus presentation device 501 is merely a display, monitor, screen, speaker, etc., that provides stimulus material to a user. Continuous and discrete modes are supported. According to various embodiments, the stimulus presentation device 501 also has protocol generation capability to allow intelligent customization of stimuli provided to multiple subjects in different markets.

According to various embodiments, stimulus presentation device 501 could include devices such as televisions, cable consoles, computers and monitors, projection systems, display devices, speakers, tactile surfaces, etc., for presenting the video and audio from different networks, local networks, cable channels, syndicated sources, websites, internet content aggregators, portals, service providers, etc.

According to various embodiments, the subjects 503 are connected to data collection devices 505. The data collection devices 505 may include a variety of neuro-response measurement mechanisms including neurographical, neurological and neurophysiological measurements systems. According to various embodiments, neuro-response data includes central nervous system, autonomic nervous system, and effector data.

Some examples of central nervous system measurement mechanisms include Functional Magnetic Resonance Imaging (fMRI), Magnetoencephalography (MEG), optical imaging, and Electroencephalography (EEG). FMRI measures blood oxygenation in the brain that correlates with increased neural activity. However, current implementations of fMRI have poor temporal resolution of few seconds. MEG measures the magnetic fields produced by electrical activity in the brain via extremely sensitive devices such as superconducting quantum interference devices (SQUIDs). optical imaging measures deflection of light from a laser or infrared source to determine anatomic or chemical properties of a material. EEG measures electrical activity associated with post synaptic currents occurring in the milliseconds range. Subcranial EEG can measure electrical activity with the most accuracy, as the bone and dermal layers weaken transmission of a wide range of frequencies. Nonetheless, surface EEG provides a wealth of electrophysiological information if analyzed properly.

Autonomic nervous system measurement mechanisms include Galvanic Skin Response (GSR), Electrocardiograms (EKG), pupillary dilation, etc. Effector measurement mechanisms include Electrooculography (EOG), eye tracking, facial emotion encoding, reaction time etc.

According to various embodiments, the techniques and mechanisms of the present invention intelligently blend multiple modes and manifestations of precognitive neural signatures with cognitive neural signatures and post cognitive neurophysiological manifestations to more accurately allow assessment of alternate media. In some examples, autonomic nervous system measures are themselves used to validate central nervous system measures. Effector and behavior responses are blended and combined with other measures. According to various embodiments, central nervous system, autonomic nervous system, and effector system measurements are aggregated into a measurement that allows definitive evaluation stimulus material

In particular embodiments, the data collection devices 505 include EEG 511, EOG 513, and FMRI 515. In some instances, only a single data collection device is used. Data collection may proceed with or without human supervision.

The data collection device 505 collects neuro-response data from multiple sources. This includes a combination of devices such as central nervous system sources (EEG, MEG, fMRI, optical imaging), autonomic nervous system sources (EKG, pupillary dilation), and effector sources (EOG, eye tracking, facial emotion encoding, reaction time). In particular embodiments, data collected is digitally sampled and stored for later analysis. In particular embodiments, the data collected could be analyzed in real-time. According to particular embodiments, the digital sampling rates are adaptively chosen based on the neurophysiological and neurological data being measured.

In one particular embodiment, the system includes EEG 511 measurements made using scalp level electrodes, EOG 513 measurements made using shielded electrodes to track eye data, functional Magnetic Resonance Imaging (fMRI) 515 measurements made non-invasively to show haemodynamic response related to neural activity, using a differential measurement system, a facial muscular measurement through shielded electrodes placed at specific locations on the face, and a facial affect graphic and video analyzer adaptively derived for each individual.

In particular embodiments, the data collection devices are clock synchronized with a stimulus presentation device 501. In particular embodiments, the data collection devices 505 also include a condition evaluation subsystem that provides auto triggers, alerts and status monitoring and visualization components that continuously monitor the status of the subject, data being collected, and the data collection instruments. The condition evaluation subsystem may also present visual alerts and automatically trigger remedial actions. According to various embodiments, the data collection devices include mechanisms for not only monitoring subject neuro-response to stimulus materials, but also include mechanisms for identifying and monitoring the stimulus materials. For example, data collection devices 505 may be synchronized with a set-top box to monitor channel changes. In other examples, data collection devices 505 may be directionally synchronized to monitor when a subject is no longer paying attention to stimulus material. In still other examples, the data collection devices 505 may receive and store stimulus material generally being viewed by the subject, whether the stimulus is a program, a commercial, printed material, or a scene outside a window. The data collected allows analysis of neuro-response information and correlation of the information to actual stimulus material and not mere subject distractions.

According to various embodiments, the system also includes a data cleanser and analyzer device 521. In particular embodiments, the data cleanser and analyzer device 521 filters the collected data to remove noise, artifacts, and other irrelevant data using fixed and adaptive filtering, weighted averaging, advanced component extraction (like PCA, ICA), vector and component separation methods, etc. This device cleanses the data by removing both exogenous noise (where the source is outside the physiology of the subject, e.g. a phone ringing while a subject is viewing a video) and endogenous artifacts (where the source could be neurophysiological, e.g. muscle movements, eye blinks, etc.).

The artifact removal subsystem includes mechanisms to selectively isolate and review the response data and identify epochs with time domain and/or frequency domain attributes that correspond to artifacts such as line frequency, eye blinks, and muscle movements. The artifact removal subsystem then cleanses the artifacts by either omitting these epochs, or by replacing these epoch data with an estimate based on the other clean data (for example, an EEG nearest neighbor weighted averaging approach).

According to various embodiments, the data cleanser and analyzer device 521 is implemented using hardware, firmware, and/or software.

The data analyzer portion uses a variety of mechanisms to analyze underlying data in the system to determine resonance. According to various embodiments, the data analyzer customizes and extracts the independent neurological and neuro-physiological parameters for each individual in each modality, and blends the estimates within a modality as well as across modalities to elicit an enhanced response to the presented stimulus material. In particular embodiments, the data analyzer aggregates the response measures across subjects in a dataset.

According to various embodiments, neurographical, neurological and neuro-physiological signatures are measured using time domain analyses and frequency domain analyses. Such analyses use parameters that are common across individuals as well as parameters that are unique to each individual. The analyses could also include statistical parameter extraction and fuzzy logic based attribute estimation from both the time and frequency components of the synthesized response.

In some examples, statistical parameters used in a blended effectiveness estimate include evaluations of skew, peaks, first and second moments, distribution, as well as fuzzy estimates of attention, emotional engagement and memory retention responses.

According to various embodiments, the data analyzer may include an intra-modality response synthesizer and a cross-modality response synthesizer. In particular embodiments, the intra-modality response synthesizer is configured to customize and extract the independent neurographical, neurological and neurophysiological parameters for each individual in each modality and blend the estimates within a modality analytically to elicit an enhanced response to the presented stimuli. In particular embodiments, the intra-modality response synthesizer also aggregates data from different subjects in a dataset.

According to various embodiments, the cross-modality response synthesizer or fusion device blends different intra-modality responses, including raw signals and signals output. The combination of signals enhances the measures of effectiveness within a modality. The cross-modality response fusion device can also aggregate data from different subjects in a dataset.

According to various embodiments, the data analyzer also includes a composite enhanced effectiveness estimator (CEEE) that combines the enhanced responses and estimates from each modality to provide a blended estimate of the effectiveness. In particular embodiments, blended estimates are provided for each exposure of a subject to stimulus materials. The blended estimates are evaluated over time to assess resonance characteristics. According to various embodiments, numerical values are assigned to each blended estimate. The numerical values may correspond to the intensity of neuro-response measurements, the significance of peaks, the change between peaks, etc. Higher numerical values may correspond to higher significance in neuro-response intensity. Lower numerical values may correspond to lower significance or even insignificant neuro-response activity. In other examples, multiple values are assigned to each blended estimate. In still other examples, blended estimates of neuro-response significance are graphically represented to show changes after repeated exposure.

According to various embodiments, a data analyzer passes data to a resonance estimator that assesses and extracts resonance patterns. In particular embodiments, the resonance estimator determines entity positions in various stimulus segments and matches position information with eye tracking paths while correlating saccades with neural assessments of attention, memory retention, and emotional engagement. In particular embodiments, the resonance estimator stores data in the priming repository system. As with a variety of the components in the system, various repositories can be co-located with the rest of the system and the user, or could be implemented in remote locations.

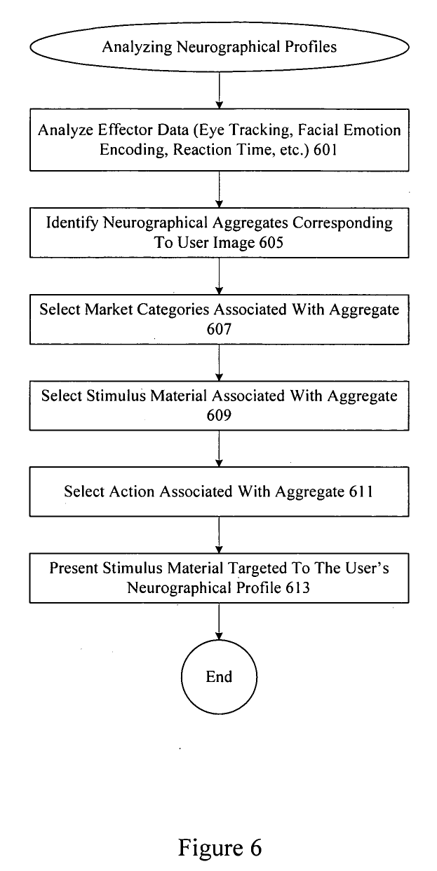

FIG. 6 illustrates an example of a technique for analyzing neurographical profiles. At 601, effector data such as eye tracking, facial emotion encoding, and reaction time data is analyzed. Autonomic nervous system measures such as pupillary dilation can also be analyzed. At 605, neurographical aggregates corresponding to a user image, video, or voice are identified. At 607, market categories associated with the neurographical aggregate are selected. At 609, stimulus material associated with the neurographical aggregate are selected. At 611, an action associated with the aggregate is determined. For example, an expression of disgust on any aggregate may trigger an action to change the stimulus material being presented.

According to various embodiments, neurographical analysis may be at times combined with neurological response analysis. For example, a volunteer may be viewing commercials while having video recorded and EEG data collected at home or at an analysis site. According to various embodiments, data analysis is performed. Data analysis may include intra-modality response synthesis and cross-modality response synthesis to enhance effectiveness measures. It should be noted that in some particular instances, one type of synthesis may be performed without performing other types of synthesis. For example, cross-modality response synthesis may be performed with or without intra-modality synthesis.

A variety of mechanisms can be used to perform data analysis. In particular embodiments, a stimulus attributes repository is accessed to obtain attributes and characteristics of the stimulus materials, along with purposes, intents, objectives, etc. In particular embodiments, EEG response data is synthesized to provide an enhanced assessment of effectiveness. According to various embodiments, EEG measures electrical activity resulting from thousands of simultaneous neural processes associated with different portions of the brain. EEG data can be classified in various bands. According to various embodiments, brainwave frequencies include delta, theta, alpha, beta, and gamma frequency ranges. Delta waves are classified as those less than 4 Hz and are prominent during deep sleep. Theta waves have frequencies between 3.5 to 7.5 Hz and are associated with memories, attention, emotions, and sensations. Theta waves are typically prominent during states of internal focus.

Alpha frequencies reside between 7.5 and 13 Hz and typically peak around 10 Hz. Alpha waves are prominent during states of relaxation. Beta waves have a frequency range between 14 and 30 Hz. Beta waves are prominent during states of motor control, long range synchronization between brain areas, analytical problem solving, judgment, and decision making. Gamma waves occur between 30 and 60 Hz and are involved in binding of different populations of neurons together into a network for the purpose of carrying out a certain cognitive or motor function, as well as in attention and memory. Because the skull and dermal layers attenuate waves in this frequency range, brain waves above 75-80 Hz are difficult to detect and are often not used for stimuli response assessment.

However, the techniques and mechanisms of the present invention recognize that analyzing high gamma band (kappa-band: Above 60Hz) measurements, in addition to theta, alpha, beta, and low gamma band measurements, enhances neurological attention, emotional engagement and retention component estimates. In particular embodiments, EEG measurements including difficult to detect high gamma or kappa band measurements are obtained, enhanced, and evaluated. Subject and task specific signature sub-bands in the theta, alpha, beta, gamma and kappa bands are identified to provide enhanced response estimates. According to various embodiments, high gamma waves (kappa-band) above 80 Hz (typically detectable with sub-cranial EEG and/or magnetoencephalograophy) can be used in inverse model-based enhancement of the frequency responses to the stimuli.

Various embodiments of the present invention recognize that particular sub-bands within each frequency range have particular prominence during certain activities. A subset of the frequencies in a particular band is referred to herein as a sub-band. For example, a sub-band may include the 40-45 Hz range within the gamma band. In particular embodiments, multiple sub-bands within the different bands are selected while remaining frequencies are band pass filtered. In particular embodiments, multiple sub-band responses may be enhanced, while the remaining frequency responses may be attenuated.

An information theory based band-weighting model is used for adaptive extraction of selective dataset specific, subject specific, task specific bands to enhance the effectiveness measure. Adaptive extraction may be performed using fuzzy scaling. Stimuli can be presented and enhanced measurements determined multiple times to determine the variation profiles across multiple presentations. Determining various profiles provides an enhanced assessment of the primary responses as well as the longevity (wear-out) of the marketing and entertainment stimuli. The synchronous response of multiple individuals to stimuli presented in concert is measured to determine an enhanced across subject synchrony measure of effectiveness. According to various embodiments, the synchronous response may be determined for multiple subjects residing in separate locations or for multiple subjects residing in the same location.

Although a variety of synthesis mechanisms are described, it should be recognized that any number of mechanisms can be applied—in sequence or in parallel with or without interaction between the mechanisms.

Although intra-modality synthesis mechanisms provide enhanced significance data, additional cross-modality synthesis mechanisms can also be applied. A variety of mechanisms such as EEG, Eye Tracking, fMRI, EOG, and facial emotion encoding are connected to a cross-modality synthesis mechanism. Other mechanisms as well as variations and enhancements on existing mechanisms may also be included. According to various embodiments, data from a specific modality can be enhanced using data from one or more other modalities. In particular embodiments, EEG typically makes frequency measurements in different bands like alpha, beta and gamma to provide estimates of significance. However, the techniques of the present invention recognize that significance measures can be enhanced further using information from other modalities.

For example, facial emotion encoding measures can be used to enhance the valence of the EEG emotional engagement measure. EOG and eye tracking saccadic measures of object entities can be used to enhance the EEG estimates of significance including but not limited to attention, emotional engagement, and memory retention. According to various embodiments, a cross-modality synthesis mechanism performs time and phase shifting of data to allow data from different modalities to align. In some examples, it is recognized that an EEG response will often occur hundreds of milliseconds before a facial emotion measurement changes. Correlations can be drawn and time and phase shifts made on an individual as well as a group basis. In other examples, saccadic eye movements may be determined as occurring before and after particular EEG responses. According to various embodiments, FMRI measures are used to scale and enhance the EEG estimates of significance including attention, emotional engagement and memory retention measures.

Evidence of the occurrence or non-occurrence of specific time domain difference event-related potential components (like the DERP) in specific regions correlates with subject responsiveness to specific stimulus. According to various embodiments, ERP measures are enhanced using EEG time-frequency measures (ERPSP) in response to the presentation of the marketing and entertainment stimuli. Specific portions are extracted and isolated to identify ERP, DERP and ERPSP analyses to perform. In particular embodiments, an EEG frequency estimation of attention, emotion and memory retention (ERPSP) is used as a co-factor in enhancing the ERP, DERP and time-domain response analysis.

EOG measures saccades to determine the presence of attention to specific objects of stimulus. Eye tracking measures the subject's gaze path, location and dwell on specific objects of stimulus. According to various embodiments, EOG and eye tracking is enhanced by measuring the presence of lambda waves (a neurophysiological index of saccade effectiveness) in the ongoing EEG in the occipital and extra striate regions, triggered by the slope of saccade-onset to estimate the significance of the EOG and eye tracking measures. In particular embodiments, specific EEG signatures of activity such as slow potential shifts and measures of coherence in time-frequency responses at the Frontal Eye Field (FEF) regions that preceded saccade-onset are measured to enhance the effectiveness of the saccadic activity data.

According to various embodiments, facial emotion encoding uses templates generated by measuring facial muscle positions and movements of individuals expressing various emotions prior to the testing session. These individual specific facial emotion encoding templates are matched with the individual responses to identify subject emotional response. In particular embodiments, these facial emotion encoding measurements are enhanced by evaluating inter-hemispherical asymmetries in EEG responses in specific frequency bands and measuring frequency band interactions. The techniques of the present invention recognize that not only are particular frequency bands significant in EEG responses, but particular frequency bands used for communication between particular areas of the brain are significant. Consequently, these EEG responses enhance the EMG, graphic and video based facial emotion identification.

According to various embodiments, post-stimulus versus pre-stimulus differential measurements of ERP time domain components in multiple regions of the brain (DERP) are measured. The differential measures give a mechanism for eliciting responses attributable to the stimulus. For example the messaging response attributable to an advertisement or the brand response attributable to multiple brands is determined using pre-resonance and post-resonance estimates

Market categories associated with the templates are selected for the user at 607. In particular embodiments, stimulus material associated with templates is selected at 609. For example, advertisements showing large gatherings of people may be selected for individuals having high extroversion levels. Advertisements having a large number of simultaneous visual elements may be selected for individuals having the capability to process a larger number of simultaneous visual elements at 611. At 613, stimulus material targeted to the neurological profile of the user is presented to the user.

According to various embodiments, various mechanisms such as the data collection mechanisms, the intra-modality synthesis mechanisms, cross-modality synthesis mechanisms, etc. are implemented on multiple devices. However, it is also possible that the various mechanisms be implemented in hardware, firmware, and/or software in a single system.

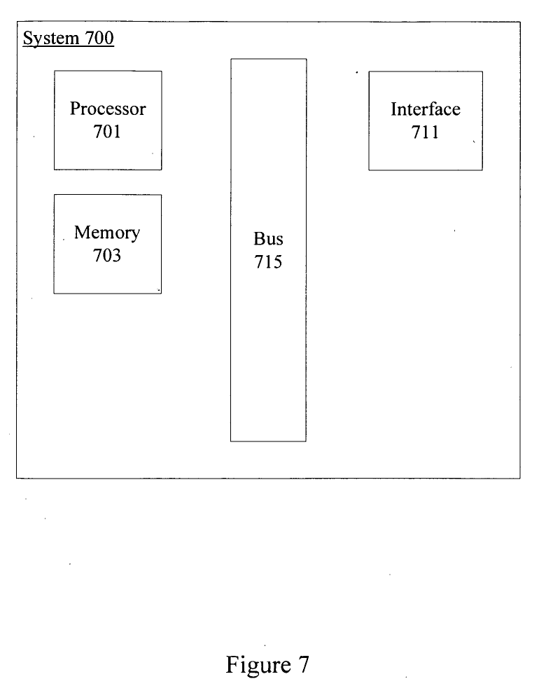

FIG. 7 provides one example of a system that can be used to implement one or more mechanisms. For example, the system shown in FIG. 7 may be used to implement a neurographical evaluation system.

According to particular example embodiments, a system 700 suitable for implementing particular embodiments of the present invention includes a processor 701, a memory 703, an interface 711, and a bus 715 (e.g., a PCI bus). When acting under the control of appropriate software or firmware, the processor 701 is responsible for such tasks such as pattern generation. Various specially configured devices can also be used in place of a processor 701 or in addition to processor 701. The complete implementation can also be done in custom hardware. The interface 711 is typically configured to send and receive data packets or data segments over a network. Particular examples of interfaces the device supports include host bus adapter (HBA) interfaces, Ethernet interfaces, frame relay interfaces, cable interfaces, DSL interfaces, token ring interfaces, and the like.

According to particular example embodiments, the system 700 uses memory 703 to store data, algorithms and program instructions. The program instructions may control the operation of an operating system and/or one or more applications, for example. The memory or memories may also be configured to store received data and process received data.

Because such information and program instructions may be employed to implement the systems/methods described herein, the present invention relates to tangible, machine readable media that include program instructions, state information, etc. for performing various operations described herein. Examples of machine-readable media include, but are not limited to, magnetic media such as hard disks, floppy disks, and magnetic tape; optical media such as CD-ROM disks and DVDs; magneto-optical media such as optical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

Although the foregoing invention has been described in some detail for purposes of clarity of understanding, it will be apparent that certain changes and modifications may be practiced within the scope of the appended claims. Therefore, the present embodiments are to be considered as illustrative and not restrictive and the invention is not to be limited to the details given herein, but may be modified within the scope and equivalents of the appended claims.

What is claimed is:

1. A method, comprising:
receiving user neurographical data;
analyzing user neurographical data using neurographical aggregates, the neurographical aggregates identifying a plurality of characteristics including an emotional expression;
identifying stimulus materials particularly effective using the user neurographical data and the neurographical aggregates;
transmitting the stimulus material for presentation to the user.

2. The method of claim 1, wherein user neurographical data is matched to a corresponding neurographical aggregate.

3. The method of claim 2, wherein the neurographical aggregate is associated with market categories and stimulus materials.

4. The method of claim 1, wherein stimulus materials are selected after determining changes in user neurographical data.

5. The method of claim 1, wherein stimulus materials are presented using a digital billboard.

6. The method of claim 1, wherein neurographical aggregates are obtained from a neurographical aggregate database.

7. The method of claim 1, wherein neurographical aggregates are generated using neuro-response data including Electroencalography (EEG).

8. The method of claim 1, wherein the plurality of characteristics include skin tone, eye color, age, and gender.

9. The method of claim 1, wherein effector data is analyzed with user neurographical data, wherein effector data includes eye tracking and reaction time.

10. The method of claim 1, wherein autonomdic nervous system data is obtained and analyzed with neurographical data, wherein autonomic nervous system data includes pupillary dilation.

11. An apparatus, comprising:
an interface configured to receive user neurographical data;
a neurographical aggregate analyzer configured to analyzing user neurographical data using neurographical aggregates, the neurographical aggregates identifying a plurality of characteristics including an emotional expression, wherein particularly effective stimulus materials are identified using the user neurographical data and the neurographical aggregates;
a stimulus presentation device configured to present stimulus material to the user.

12. The apparatus of claim 11, wherein user neurographical data is matched to a corresponding neurographical aggregate.

13. The apparatus of claim 12, wherein the neurographical aggregate is associated with market categories and stimulus materials.

14. The apparatus of claim 11, wherein stimulus materials are selected after determining changes in user neurographical data.

15. The apparatus of claim 11, wherein stimulus materials are presented using a digital billboard.

16. The apparatus of claim 11, wherein neurographical aggregates are obtained from a neurographical aggregate database.

17. The apparatus of claim 11, wherein neurographical aggregates are generated using neuro-response data including Electroencalography (EEG).

18. The apparatus of claim 11, wherein the plurality of characteristics include skin tone, eye color, age, and gender.

19. The apparatus of claim 11, wherein effector data is analyzed with user neurographical data, wherein effector data includes eye tracking and reaction time.

20. The apparatus of claim 11, wherein autonomdic nervous system data is obtained and analyzed with neurographical data, wherein autonomic nervous system data includes pupillary dilation.

21. A system, comprising:
means for receiving user neurographical data;
means for analyzing user neurographical data using neurographical aggregates, the neurographical aggregates identifying a plurality of characteristics including an emotional expression;
means for identifying stimulus materials particularly effective using the user neurographical data and the neurographical aggregates;
means for transmitting the stimulus material for presentation to the user.
